SEQUENCE LISTING (2 sequences)

SEQ ID NO: 1 - DNA, 5967 bp, Homo sapiens
  CDS (191)..(1633)
ctttgggcctcataaacaaccacagaaccacaagttgggtagcctggcagtgtcagaagtctgaacccagcatagtggtcagcaggcaggacgaatcacactgaatgcaaaccacagggtttcgcagcgtggtaaaagaaatcattgagtcccccgccttcagaagagggtgcattttcaggaggaagcgatggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 2 - protein, 480 aa, Homo sapiens
MASDSIFESFPSYPQCFMRECILGMNPSRDVHDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

(19) United States
(12) Patent Application Publication
(10) Pub. No.: US 2019/0008980 A1
(43) Pub. Date: Jan. 10, 2019

(54) POLYION COMPLEX CAPABLE OF EFFICIENTLY DELIVERING MRNA INTO LIVING BODY, AND DRUG AND METHOD FOR TREATING ARTHROPATHY IN WHICH SAID COMPLEX IS USED

(71) Applicant: AccuRna, Inc., Bunkyo-ku Tokyo (JP)

(72) Inventors: Keiji ITAKA, Tokyo (JP); Shinsuke OHBA, Tokyo (JP); Yuichi TEI, Tokyo (JP); Kazunori KATAOKA, Tokyo (JP)

(73) Assignee: AccuRna, Inc., Bunkyo-ku Tokyo (JP)

(21) Appl. No.: 15/748,767

(22) PCT Filed: Jul. 29, 2016
(86) PCT No.: PCT/JP2016/072322

§ 371 Date: Jan. 30, 2018

(30) Foreign Application Priority Data
Jul. 31, 2015 (JP) 2015-151564

(51) Int. Cl. A61K 47/64; A61K 31/7105; A61K 47/60; A61K 47/69; A61K 9/00; A61P 19/02

(52) U.S. Cl. CPC A61K 47/6455; A61K 31/7105; A61P 19/02; A61K 47/6907; A61K 9/0019; A61K 47/60

(57) ABSTRACT

The present invention provides a polyion complex which can efficiently deliver mRNA into a living body as well as a therapeutic agent and a therapeutic method of arthropathy in which the polyion complex is used. For example, there is provided a polyion complex comprising a cationic polymer and mRNA, wherein the cationic polymer is a polymer comprising a cationic unnatural amino acid as a monomer unit and the cationic unnatural amino acid is an amino acid having a group represented by —(NH—(CH₂)₂)ₚ—NH₂, wherein p is 2, 3 or 4, as a side chain.

### CROSS-REFERENCE TO RELATED APPLICATIONS

This application claims the benefit of priority of Japanese Patent Application No. 2015-151564 filed on Jul. 31, 2015, the content of which is incorporated herein by reference in its entirety.

### TECHNICAL FIELD

The present invention relates to a polyion complex which can efficiently deliver mRNA into a living body as well as a therapeutic agent and a therapeutic method of arthropathy in which the polyion complex is used.

### BACKGROUND ART

Drug delivery systems for delivering drugs to a suitable site in a body have been researched and developed to provide a new medicament with few side effects. Among these systems, a drug delivery system using a polyion complex (hereinafter also referred to as “PIC”) is drawing attention as a technique which can specifically deliver drugs to affected areas by including the drugs in nanomicelles.

As a technique applying PIC, in particular, a system which can deliver nucleic acids into a body with few side effects has been developed (Patent Literature 1 and 2). Inventors in Patent Literature 1 and 2 have developed a polyion complex of DNA and a new cationic polymer. A polyion complex of RNA and a cationic polymer has also been published by inventors (Non Patent Literature 1).

Osteoarthritis (OA) is a chronic degradative joint disease and is caused by collapse of balance between synthesis and degradation of the cartilage (Non Patent Literature 2). Osteoarthritis is a major health problem in the elderly (Non Patent Literature 3). However, disease-modifying drugs for osteoarthritis (DMOAD) for clinical use have not been developed yet (Non Patent Literature 4 and 5).

Runx1 is known to control chondrogenesis in embryos and adults (Non Patent Literature 6 and 7). Two different DMOAD candidate compounds have been proved to have therapeutic effects through Runx1 (Non Patent Literature 8 and 9).

### CITATION LIST

### Patent Literature

Patent Literature 1: Japanese Patent No. 4535229
Patent Literature 2: Japanese Patent No. 5061349

### Non Patent Literature

Non Patent Literature 1: H. Uchida et al., Journal of the American Chemical Society 136, 12396-12405 (2014)
Non Patent Literature 2: G. Nuki, Z Rheumatol, 58, 142-147 (1999)
Non Patent Literature 3: D. T. Felson et al., Annals of Internal Medicine 133, 635-646 (2000)
Non Patent Literature 4: Z. Jotanovic et al., Current drug targets 15, 635-661 (2014)
Non Patent Literature 5: D. J. Hunter, Nat Rev Rheumatol 7, 13-22 (2011)
Non Patent Literature 6: A. Kimura et al., Development. (England, 2010), vol. 137, pp. 1159-1167
Non Patent Literature 7: K. T. LeBlanc et al., J. Cell. Physiol., 230 (2), pp. 440-448 (2015)
Non Patent Literature 8: F. Yano et al., Ann Rheum Dis 72, 748-753 (2013)
Non Patent Literature 9: K. Johnson et al., Science 336, 717-721 (2012)

### SUMMARY OF INVENTION

### Technical Problem

The present invention provides a polyion complex which can efficiently deliver mRNA into a living body as well as a therapeutic agent and a therapeutic method of arthropathy in which the polyion complex is used.

### Solution to Problem

The present inventors proved that a polyion complex comprising a cationic polymer and mRNA, wherein the cationic polymer is a polymer comprising an unnatural amino acid as a monomer unit and the unnatural amino acid is an amino acid having a group represented by —(NH—(CH₂)₂)ₚ—NH₂, wherein p is 2, 3 or 4, as a side chain, highly expresses mRNA continuously for a long time. The present inventors have proved that osteoarthritis can be treated by administering the polyion complex comprising the cationic polymer and Runx1 mRNA. The present invention is based on these findings.

According to the present invention, following inventions are provided.

(1) a polyion complex comprising a cationic polymer and mRNA,
wherein the cationic polymer is a polymer comprising a cationic unnatural amino acid as a monomer unit and the cationic unnatural amino acid is an amino acid having a group represented by —(NH—(CH₂)₂)ₚ—NH₂, wherein p is 2, 3 or 4, as a side chain.
(2) The polyion complex according to (1) above, wherein the cationic polymer is a block copolymer with polyethylene glycol.
(3) The polyion complex according to (1) or (2) above, wherein the cationic polymer is represented by the following general formula (I):

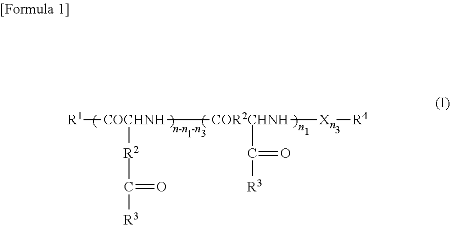

wherein
R¹ is polyethylene glycol and may be linked to an amino acid neighboring the polyethylene glycol through a linker,
R² is methylene or ethylene,
R³ is a group represented by —(NH—(CH₂)₂)ₚ—NH₂, and p is 2, 3 or 4,
R⁴ is hydrogen, a protecting group, a hydrophobic group, or a polymerizable group,
X is a cationic natural amino acid,
n is any integer from 2 to 5000,
n₁ is any integer from 0 to 5000,
n₃ is any integer from 0 to 5000,
n−n₁−n₃ is an integer equal to or greater than 0, and though the repeating units in the formula are shown in a particular order for convenience of description, the repeating units may be present in any order and the repeating units may be present at random, and each of the repeating units may be same or different.
(4) A pharmaceutical composition for treating arthropathy, comprising the polyion complex according to any one of (1) to (3) above, wherein the mRNA is a Runx1 mRNA.
(5) The pharmaceutical composition according to (4) above, wherein p is 3 or 4.
(6) The pharmaceutical composition according to (5) above, wherein p is 4.
(7) The pharmaceutical composition according to any one of (4) to (6) above, wherein the pharmaceutical composition is administered once every 2 days or once every 3 days.
(8) The pharmaceutical composition according to (5) or (6) above, wherein the pharmaceutical composition is administered once every 7 days.
(9) The pharmaceutical composition according to any one of (4) to (8) above, wherein the arthropathy is osteoarthritis or rheumatoid arthritis.
(10) A delivery agent for use in delivering mRNA into cells, comprising the polyion complex according to (3) above, wherein p is 3 or 4.
(11) The delivery agent according to (10) above, wherein p is 4.

### BRIEF DESCRIPTION OF DRAWINGS

FIG. 1 is photos showing the expression of luciferase mRNA intraarticularly administered.

FIG. 2 is graphs showing the transition over time of the expression level of luciferase mRNA intraarticularly administered.

FIG. 3 shows the effect of administration of Runx1 mRNA on osteoarthritis. A polymer in which p is 2 is used as a cationic polymer in FIG. 3.

FIG. 4 shows the effect of administration of Runx1 mRNA on osteoarthritis. A polymer in which p is 3 is used as a cationic polymer in FIG. 4.

FIG. 5 shows the expression of various marker genes and the effect on cell death examined by TUNEL method in group receiving administration of Runx1 mRNA.

### DESCRIPTION OF EMBODIMENT

The polyion complex of the present invention comprises at least (i) a block copolymer comprising a cationic polymer and (ii) mRNA. The polyion complex of the present invention is characterized in that the cationic polymer is a polymer comprising an unnatural amino acid as a monomer unit and the unnatural amino acid has a group represented by —(NH—(CH₂)₂)ₚ—NH₂, wherein p is 2, 3 or 4, as a side chain. P is preferably 3 or 4, and most preferably 3. The cationic polymer may be a cationic polymer forming a block copolymer with a polyethylene glycol block. (i) the copolymer and (ii) the mRNA form a polyion complex in a solution. The polyion complex of the present invention can be provided in a solution, preferably in an aqueous solution.

The polyion complex of the present invention comprises a cationic polymer and mRNA, and is thought to be in the form of nanomicelles. Therefore, the polyion complex of the present invention may be referred to as a polyion complex-type micelle in the specification.

As used herein, mRNA refers to messenger RNA.

Cytidine and uridine in mRNA in the polyion complex of the present invention may be modified. Examples of the modified cytidine include, for example, 5-methyl-cytidine, and examples of the modified uridine include pseudouridine and 2-thio-cytidine. The modified cytidine and uridine may comprise 10 mole % or more, 20 mole % or more, or 30 mole % or more of total cytidine and uridine.

As used herein, “a subject” is a mammal including a human. The subject may be a healthy subject or may be a subject suffering from some disease.

In the present invention, the cationic polymer may comprise, for example, a cationic natural amino acid and/or a cationic unnatural amino acid as a monomer unit. Here, examples of the cationic natural amino acid preferably include histidine, tryptophane, ornithine, arginine and lysine, more preferably include arginine, ornithine and lysine, further preferably include ornithine and lysine, and further more preferably include lysine.

In the present invention, the polymer part comprising an unnatural amino acid as a monomer unit in the cationic polymer can be obtained, for example, by introducing a group represented by —(NH—(CH₂)₂)ₚ—NH₂, wherein p is 2, 3 or 4, into a polymer comprising aspartic acid or glutamic acid as a monomer unit. Those skilled in the art can obtain this polymer part comprising the unnatural amino acid as a monomer unit by reacting a polymer comprising aspartic acid or glutamic acid as a monomer unit with diethyltriamine, triethyltetraamine or tetraethylpentaamine.

In the preferred embodiment of the present invention, the cationic polymer can be a polymer represented by the following general formula (I):

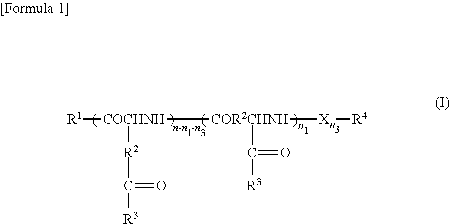

wherein
R¹ is polyethylene glycol and may be linked to an amino acid neighboring the polyethylene glycol through a linker,
R² is methylene or ethylene,
R³ is a group represented by —(NH—(CH₂)₂)ₚ—NH₂, and p is 2, 3 or 4,
R⁴ is hydrogen, a protecting group, a hydrophobic group, or a polymerizable group,
X is a cationic amino acid,
n is any integer from 2 to 5000,
n₁ is any integer from 0 to 5000,
n₃ is any integer from 0 to 5000,
n−n₁−n₃ is an integer equal to or greater than 0, and though the repeating units in the formula are shown in a particular order for convenience of description, the repeating units may be present in any order and the repeating units may be present at random, and the repeating units may be same or different.

In the formula (I) above, p is preferably 3 or 4, and most preferably 3.

In the formula (I) above, examples of the protecting group include C₁₋₆ alkylcarbonyl groups, and the protecting group is preferably an acetyl group, and examples of the hydrophobic group include benzene, naphthalene, anthracene, pyrene and derivatives thereof or C₁₋₆ alkyl groups, and examples of the polymerizable group include a methacryloyl group and an acryloyl group. Methods for introducing the protecting group, the hydrophobic group and the polymerizable group into a block copolymer are well-known to those skilled in the art.

In the formula (I) above, though the average degree of polymerization of polyethylene glycol (PEG) is 5 to 20000, preferably 10 to 5000, and more preferably 40 to 500, the degree is not particularly limited as long as the polyion complex formation of the block copolymer and mRNA is not inhibited. The end of PEG of the cationic polymer may be protected by a hydroxy group, a methoxy group or a protecting group.

In the formula (I) above, the linker may be for example —(CH₂)r-NH—, wherein r is any integer from 1 to 5, or —(CH₂)s-CO—, wherein s is any integer from 1 to 5, and may be linked to the neighboring amino acid in the formula (I) preferably through a peptide bond. The linker may be preferably linked to PEG on the methylene side of PEG through an O atom of PEG.

In the formula (I) above, n is any integer from 0 to 5000, for example, any integer from 0 to 500; m is any integer from 0 to 5000, for example, any integer from 0 to 500; and m+n is any integer from 2 to 5000, for example, any integer from 2 to 500.

In one embodiment of the present invention, the cationic polymer may be a block copolymer of a PEG-linker-polycation block, where the PEG, the linker and the polycation block are as defined above.

The present inventors have found that when the cationic polymer used for the formation of a polyion complex is a polymer comprising a monomer unit having a group represented by —(NH—(CH₂)₂)ₚ—NH₂, wherein p is 2, 3 or 4, as a side chain, mRNA can be present in a body for a long time, and that, in particular, when p is 3 or 4, mRNA is highly expressed continuously for a considerably long time in a body. Therefore, in the present invention, the cationic polymer may be a cationic polymer comprising a monomer unit having a group represented by —(NH—(CH₂)₂)ₚ—NH₂, wherein p is 2, 3 or 4, as a side chain, wherein p is more preferably 3 or 4, and further preferably 3.

The present inventors administered the polyion complex of the present invention using a Runx1 mRNA as the mRNA in the polyion complex to arthropathy model animals and have found that the progress of symptoms of arthropathy was slowed or the symptoms were improved by Runx1.

Therefore, in the present invention, a pharmaceutical composition for use in treating arthropathy comprising the polyion complex of the present invention is provided. In one embodiment of the present invention, the mRNA is an mRNA of a factor promoting joint formation. Non-limiting examples of the factor promoting joint formation include transcription factors promoting joint formation, such as Runt-related transcription factor 1 (Runx1), core-binding factor beta (Cbfbeta), sex-determining box 9 (Sox9) and Proteoglycan 4.

In one embodiment of the present invention, arthropathy is osteoarthritis or rheumatoid arthritis.

In one embodiment of the present invention, the pharmaceutical composition is a pharmaceutical composition for use in treating arthropathy (for example, osteoarthritis or rheumatoid arthritis), comprising polyion complex-type micelles of a cationic polymer and a Runx1 mRNA, wherein R¹ in the formula (I) is protected polyethylene glycol, and p is 3 or 4, and R⁴ is hydrogen or a protecting group.

In one embodiment, the pharmaceutical composition of the present invention may further comprise an excipient.

In one embodiment, the pharmaceutical composition of the present invention may be formulated for intraarticular administration.

In another aspect of the present invention, there is provided a method for treating arthropathy in a subject in need thereof, comprising administering the effective amount of the pharmaceutical composition of the present invention to a subject.

In the present invention, the administration of the pharmaceutical composition of the present invention to a subject allows the expression of a protein from mRNA to be sustained in the subject at least for 2 days, preferably for 3 days. Therefore, the pharmaceutical composition of the present invention can be administered once every 2 days or once every 3 days.

When p is 3 or 4 in the group represented by —(NH—(CH₂)₂)ₚ—NH₂, mRNA can be expressed in a subject at least for 4 days, preferably for 8 days. Therefore, when p is 3 or 4 in the group represented by —(NH—(CH₂)₂)ₚ—NH₂, the pharmaceutical composition of the present invention can be administered, for example, once every 3 days, preferably once every 4 days, more preferably once every 5 days, further preferably once every 6 days, and further more preferably once every week. Thereby, frequency of administration is decreased and a patient's burden of treatment is greatly reduced.

### EXAMPLES

### Example 1: Production of a Polyion Complex

First, a polyion complex containing mRNA was produced.

### 1-1. Synthesis of a Polymer Comprising pAsp (DET), pAsp (TET) or pAsp (TEP) as a Monomer Unit

In this Example, the group represented by —(NH—(CH₂)₂)ₚ—NH₂, wherein p is 2, 3 or 4, was introduced to a side chain of polyaspartic acid to obtain the title polymer. Specifically, polyethylene glycol with a number average molecular weight of 23,000 having a methoxy group at one end and an aminopropyl group at the other end (MeO-PEG-NH₂) (purchased from NOF CORPORATION) was dissolved in methylene chloride. β-benzyl-L-aspartate-N-carboxy anhydride (BLA-NCA) (purchased from Chuo Kaseihin Co., Inc.) was added to and dissolved in a mixed solution of N,N-dimethylformamide (DMF) and the methylene chloride solution to obtain a reaction solution. Then, the reaction solution was reacted at 40° C. for 2 days to obtain polyethylene glycol-poly (β-benzyl-L-aspartate) block copolymer (MeO-PEG-PBLA). Analysis by ¹H-NMR showed that the number average molecular weight of the PBLA moiety was about 14,000 and the degree of polymerisation was about 66.

Next, fractions of MeO-PEG-PBLA were reacted with diethylenetriamine, triethyltetraamine and tetraethylpentaamine, respectively, to obtain MeO-PEG-pAsp (DET) block copolymer, MeO-PEG-pAsp (TET) block copolymer, and MeO-PEG-pAsp (TEP) block copolymer. Specifically, MeO-PEG-PBLA was dissolved in benzene and the resultant was freeze-dried. The freeze-dried MeO-PEG-PBLA was dissolved in N,N-dimethylformamide (DMF). Thereafter, diethylenetriamine, triethyltetraamine and tetraethylpentaamine (all purchased from Wako Pure Chemical Industries, Ltd.) were dropped to the obtained solutions, respectively, and the mixtures were reacted under mild anhydrous conditions of 40° C. to obtain MeO-PEG-pAsp (DET) block copolymer, MeO-PEG-pAsp (TET) block copolymer, and MeO-PEG-pAsp (TEP) block copolymer. The obtained block copolymers showed sharp single molecular weight distribution (Mw/Mn=1.04) in gel permeation chromatography (GPC).

### 1-2. Preparation of mRNA

First, human RUNX1 expression vectors were prepared. An open reading frame of 3×FLAG tagged human RUNX1 (GenBank registration number: NM_001754.4; SEQ ID NO: 1) flanked with XhoI and BaII/SmaI restriction enzyme cleavage sites (hereinafter, referred to as RUNX1-FL) was cloned into pUC57 vectors. The obtained RUNX1-FLs were introduced into pSP17 vectors. Driven by T7 promoters, human RUNX1-FLs were transcribed from lineared pSP17 vectors using mMESSAGE mMACHINE T7 Ultra Kit (Ambion, Carlsbad, Calif., USA), and then poly A was added to the transcripts using a poly (A) tail kit (Ambion). The transcribed mRNAs were purified using RNeasy Mini Kit (Qiagen, Hilden, Germany). EGFP mRNA was prepared in the same way. Luc2 mRNA was prepared using the transcriptional region of a protein of pGL4.13 (Promega, Madison, Wis., USA) in the same way.

### 1-3. Preparation of Polyion Complex-Type Micelles

Each of the copolymer solutions obtained in 1-1 and various mRNA solutions were mixed separately in 10 mM Hepes buffer (pH7.3) to obtain polyion complexes. The concentration of mRNAs was 225 μg/mL, and the mixing ratio (N/P) of an amino group (N) of amino acid residues in the copolymers and a phosphoric acid group (P) in nucleic acids was 8. Because the copolymer solutions and the mRNA solutions were mixed in a quantitative ratio of 1:2, the concentration of mRNA became 150 μg/mL.

As a control, a lipoplex of mRNA was prepared according to a manufacturer's manual using Lipofectamine™ 2000 (Life technologies, Carlsbad, Calif.).

### Example 2: Intraarticular Administration Test of Polyion Complex-Type Micelles Comprising mRNA

20 μL of polyion complex-type micelles prepared to comprise 3 μg of Luc2 was administered to the normal knee joints of ICR mice (8 week old) under anesthesia. 100 μL of a solution comprising 1.5 mg D-luciferin was intravenously administered to the mice, and the expression of luciferase in the joints was examined at various time points (24 hours, 48 hours, or 96 hours after the administration of micelles) using IVIS™ Imaging System (Xenogen, Alameda, Calif., US).

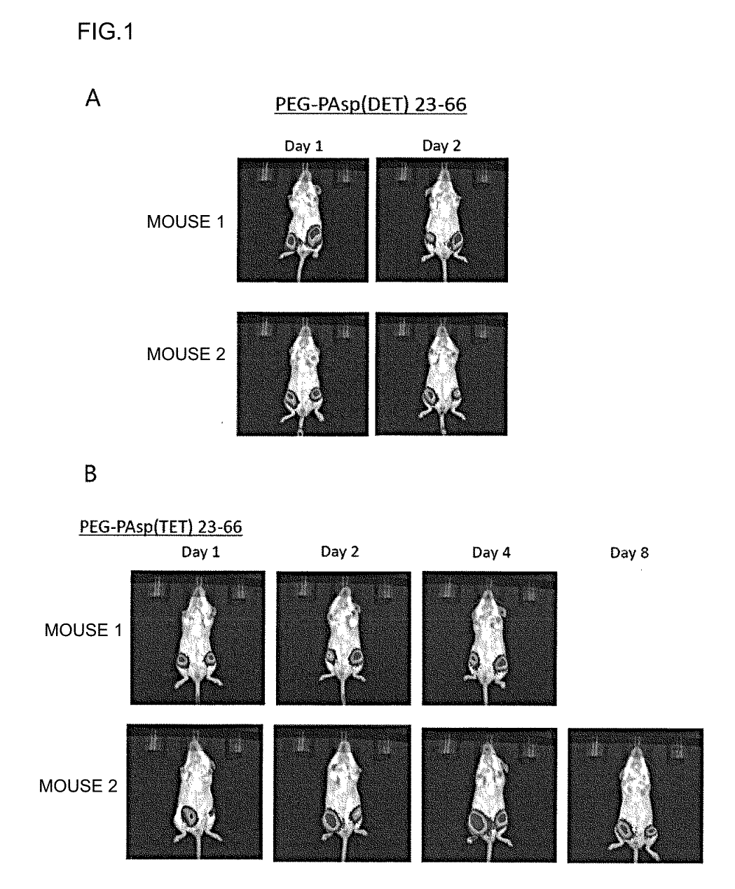

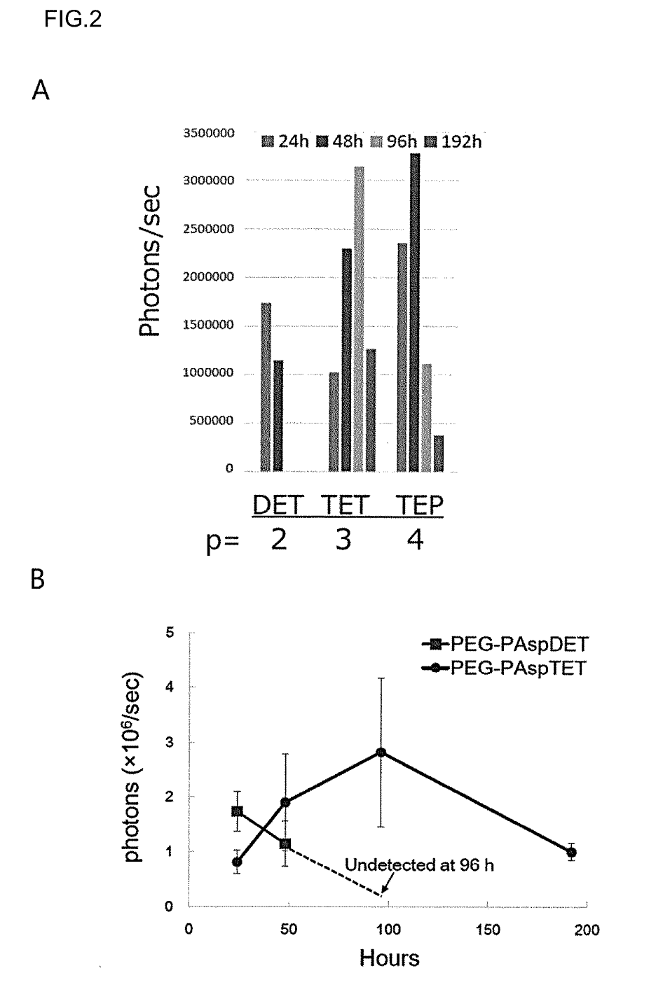

The expression of luciferase in the joints could be observed within one day after the intraarticular administration (FIGS. 1 and 2).

When PEG-PAsp (DET) was used as a cationic polymer, though the expression level decreased, the expression of luciferase was confirmed also 2 days after the administration (FIG. 1A).

When PEG-PAsp (TET) was used as a cationic polymer, the expression was sustained for at least 4 days and the expression level was also maintained (FIG. 1B). Though the expression level was decreased, the expression of luciferase was confirmed also 8 days after the administration (FIG. 1B). For PEG-PAsp (TEP), photos are not shown, but the expression was sustained for at least 4 days as the same as PEG-PAsp (TET) and the expression of luciferase was confirmed even 8 days after the administration (data not shown).

The photon count per second was calculated from the obtained results to quantify the expression level of luciferase (FIG. 2). According to FIGS. 2A and B, PEG-PAsp (DET) sustained the expression of mRNA for at least 2 days. PEG-PAsp (TET) highly expressed luciferase from one day after the administration, and surprisingly, it increased the expression level 2 days and 4 days after the administration, and sustained the expression for at least 8 days after the administration (FIGS. 2A and B). Further, PEG-PAsp (TEP) highly expressed luciferase 1 to 2 days after the administration, and then decreased the expression level, but sustained the expression for 8 days after the administration (FIG. 2A).

Thus, all of PEG-PAsp (DET), PEG-PAsp (TET) and PEG-PAsp (TEP) could highly express mRNA in a living body for at least 2 days. PEG-PAsp (TET) and PEG-PAsp (TEP) could highly express mRNA in a living body for at least 8 days.

Next, the delivery of mRNA to the cartilages by the cationic polymers used in the Example and the presence or absence of toxicity caused by the delivery were examined.

20 μL of polyion complex-type micelles comprising EGFP mRNA (3 μg) and PEG-PAsp (DET) was intraarticularly administered to normal knees of mice. As a control, 20 μL of phosphate buffer solution (PBS) was intraarticularly administered to normal knees of mice in the same way. The knees were subjected to a histological analysis 24 hours after the administration.

Histological sections of the knees were prepared, and the expression of EGFP mRNA in the knee joints was immunohistologically examined using anti-GFP antibodies. Then, EGFP protein was expressed in the surface and the central part of the articular cartilage. The degree of cell death in the articular cartilage was conventionally observed using TUNEL method, which proved that there was no difference between the polyion complex-type micelle treated group and the PBS treated group. Thus, it was proved that the polyion complex-type micelle used in this Example could express mRNA in a living body (for example, in a knee joint) and that the administration in an amount allowing the expression of mRNA did not exhibit toxicity. Though mRNA is known to have high immunogenicity, problems resulting from the antigenicity of mRNA were not seen as far as we observed.

### Example 3: Treatment of Osteoarthritis in Osteoarthritis Model Animals

To date, delivery of mRNA into a living body and sustained expression thereof have been difficult, and thus there were high barriers for the treatment of diseases in a living body. According to Example 2, use of the polyion complex of the present invention allowed mRNA to be highly expressed in a living body sustainably for a long time. Therefore, in this Example, treatment of diseases using the polyion complex of the present invention was attempted.

As osteoarthritis model animals, osteoarthritis model mice were used. These model mice were prepared by surgically removing medial collateral ligaments and medial menisci of the knees (refer to S. Kamekura et al., Osteoarthritis Cartilage 13, 632-641 (2005)). Osteoarthritis was induced one month after the surgery. Seriousness of osteoarthritis was evaluated by the histological OARSI scoring system (see S. S. Glasson et al., 2010 Osteoarthritis Research Society International. Published by Elsevier Ltd, England, vol. 18 Suppl 3, pp. S17-23 (2010)).

Runx1 was selected as a factor promoting joint formation, and Runx1 mRNA and PEG-PAsp (DET) were mixed to form polyion complex-type micelles as described in Example 1. 20 μL of the obtained micelle solution (i.e., 3 μg as Runx1 mRNA) was administered to knees of the osteoarthritis mice one month after the surgery at a frequency of once every 3 days for one month. As a negative control, the polyion complex-type micelles in which EGFP was used as mRNA were administered to knees of the osteoarthritis mice one month after the surgery at a frequency of once every 3 days for one month.

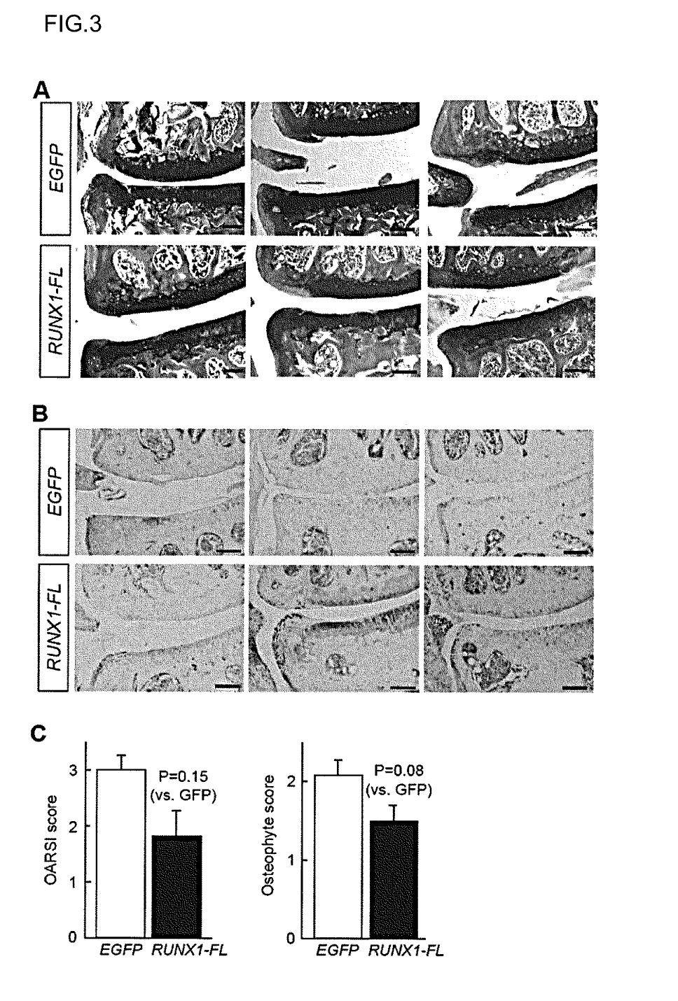

One month after the administration, typical symptoms of osteoarthritis were observed in tissue sections stained with safranin —O of the negative control. Observed symptoms included osteophyte and cartilage degradation, and these were proved by the reduction of the thickness of articular cartilage, staining intensity, and the destruction of the structure of articular cartilage (FIG. 3A). On the other hand, in the group to which Runx1 mRNA was introduced, the phenotype of osteoarthritis tended to be inhibited, and in particular, the inhibition was significant in the osteophyte formation (FIG. 3A).

The expression of the Runx1 protein in the knees two months after the surgery (i.e. one month after the completion of administration) was immunohistologically examined. The mice were euthanized and were fixed by circulating PBS including 4% paraformaldehyde. The knee joints were collected and further fixed overnight with PBS including 4% paraformaldehyde, and decalcified with PBS including 0.5 M ethylenediaminetetraacetic acid (EDTA). Then, the knee joints were embedded with paraffin to produce front sections with a thickness of 7 μm.

The sections were stained by combining an anti-Flag antibody (1:100; F1804, M2, Sigma-Aldrich), an anti-GFP antibody (1:500; ab290, Abcam, Cambridge, Mass.), an anti-Runx1 antibody (1:100; ab23980, Abcam), an anti-Sox9 antibody (1:200; sc20095, Santa Cruz Biotechnology), and an anti-PCNA antibody (1:1000; 2586s, Cell Signaling Technology) with CSAII Biotin-free Tyramide Signal Amplication System (Dako, Glostrup, Denmark). The sections were also stained with an anti-type X collagen antibody (1:500; LB-0092, LSL-Cosmo Bio co., ltd., Tokyo, Japan), an anti-type II collagen antibody (1:500; LB-1297, LSL-Cosmo Bio co., ltd.), an anti-IL-1β antibody (1:100; sc7884, Santa Cruz Biotechnology), and an anti-MMP-13 antibody (1:500; mab13426, Millipore). All sections were counterstained with methyl green.

The result showed that the Runx1 protein was strongly expressed in the articular cartilage and the expression was particularly significant in the osteophyte-like region (FIG. 3B). The evaluation of the seriousness of arthropathy proved that the seriousness of group receiving administration of Runx1 mRNA decreased compared to the negative control group (FIG. 3C).

Next, treatment of osteoarthritis was attempted as same as described above except that PEG-PAsp (TET) was used instead of PEG-PAsp (DET) as a cationic polymer.

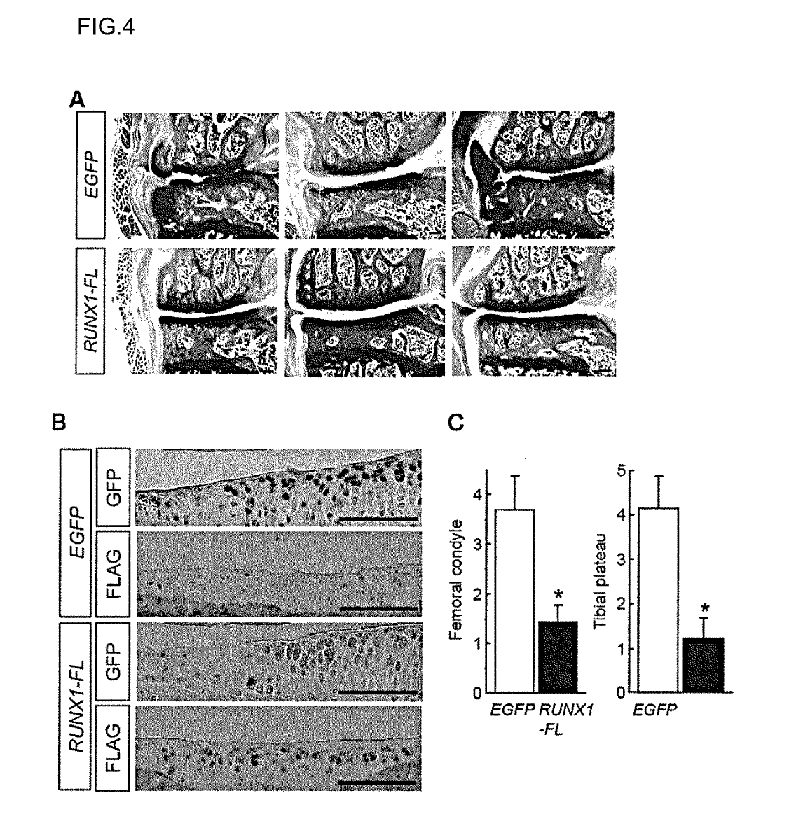

It was proved from the sections stained with safranin —O that osteoarthritis was inhibited in the group receiving administration of Runx1 mRNA compared to the EGFP-administered control group (FIG. 4A). Expression of the Runx1 protein was strongly observed in the articular cartilage (data not shown). Further, the result of immunohistological observation showed that EGFP and Runx1 proteins were strongly expressed in the articular cartilage (FIG. 4B). Osteoarthritis and the formation of osteophyte were statistically significantly inhibited in the group receiving administration of Runx1 mRNA compared to the negative control group (p<0.05) (FIG. 4C).

Further, expression levels of various factors were examined in the group receiving administration of Runx1 mRNA and the negative control group.

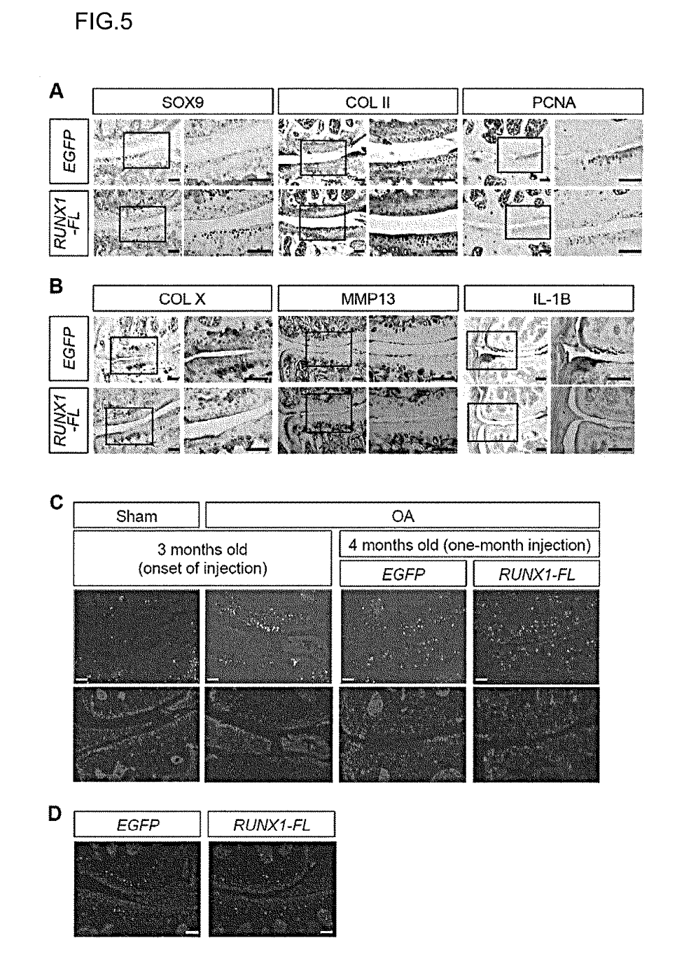

Sox9 is a master transcription factor of differentiation into cartilage and maintenance thereof in embryos and adults, and it was more strongly expressed in articular chondrocytes of the group receiving administration of Runx1 mRNA than the negative control group (upper left panels of FIG. 5A). Type II collagen is a major cartilage matrix protein, and it was more strongly expressed in the group receiving administration of Runx1 mRNA than the negative control group (upper middle panels of FIG. 5A). Further, proliferating cell nuclear antigen (PCNA) was also more strongly expressed in the group receiving administration of Runx1 mRNA than the negative control group (upper right panels of FIG. 5). These results suggest that Runx1 has a function which promotes the early differentiation of chondrocytes.

On the other hand, the expression of type X collagen and MMP13 was not significantly different between the group receiving administration of Runx1 mRNA and the negative control group (lower left panels and lower middle panels of FIG. 5). The expression level of IL-1β was lower in the group receiving administration of Runx1 mRNA than the negative control group (lower right panels of FIG. 5).

Influence of administration of Runx1 mRNA on apoptosis was analyzed by TUNEL method. However, the cell death in the joints of osteoarthritis was not significantly different between the group receiving administration of the mRNA of Runx1 and the negative control group (FIGS. 5C and D).

Relation between rheumatoid arthritis and Runx1 is known and it is also known that pathological conditions are mediated by IL-β in rheumatoid arthritis. In this Example, Runx1 significantly inhibited the expression of IL-1β in the joints, and thus it is apparent that rheumatoid arthritis can also be treated by the method of this Example.

1: A polyion complex comprising a cationic polymer and mRNA,
wherein the cationic polymer is a polymer comprising a cationic unnatural amino acid as a monomer unit and the cationic unnatural amino acid is an amino acid having a group represented by —(NH—(CH₂)₂)ₚ—NH₂, wherein p is 2, 3 or 4, as a side chain.

2: The polyion complex according to claim 1, wherein the cationic polymer is a block copolymer with polyethylene glycol.

3: The polyion complex according to claim 1, wherein the cationic polymer is represented by formula (I): 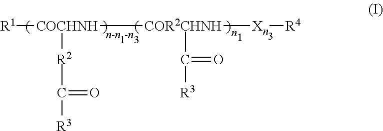
wherein
R¹ is polyethylene glycol and may be linked to an amino acid neighboring the polyethylene glycol through a linker,
R² is methylene or ethylene,
R³ is a group represented by —(NH—(CH₂)₂)ₚ—NH₂, and p is 2, 3 or 4,
R⁴ is hydrogen, a protecting group, a hydrophobic group, or a polymerizable group,
X is a cationic natural amino acid,
n is any integer from 2 to 5000,
n₁ is any integer from 0 to 5000,
n₃ is any integer from 0 to 5000,
n−n₁−n₃ is an integer equal to or greater than 0, and
though the repeating units in the formula are shown in a particular order for convenience of description, the repeating units may be present in any order and the repeating units may be present at random, and each of the repeating units may be the same or different.

4: A pharmaceutical composition for treating arthropathy, comprising the polyion complex according to claim 1,
wherein the mRNA is an mRNA of a factor promoting joint formation.

5: The pharmaceutical composition according to claim 4, wherein p is 3 or 4.

6: The pharmaceutical composition according to claim 5, wherein p is 4.

7: The pharmaceutical composition according to claim 4, wherein the pharmaceutical composition is formulated for administration once every 2 days or once every 3 days.

8: The pharmaceutical composition according to claim 5, wherein the pharmaceutical composition is formulated for administration once every 7 days.

9: The pharmaceutical composition according to claim 4, wherein the arthropathy is osteoarthritis or rheumatoid arthritis.

10: A delivery agent suitable for use in delivering mRNA into cells, comprising the polyion complex according to claim 3, wherein p is 3 or 4.

11: The delivery agent according to claim 10, wherein p is 4.
